(19) United States
(12) Patent Application Publication
(10) Pub. No.: US 2026/0072503 A1
(43) Pub. Date: Mar. 12, 2026

(54) ELECTRODE ARRAY LONGEVITY

(71) Applicant: Precision Neuroscience Corporation, New York, NY (US)

(72) Inventors: Stephanie RIDER, New York, NY (US); Mark Hettick, Fremont, CA (US)

(73) Assignee: PRECISION NEUROSCIENCE CORPORATION, New York, NY (US)

(21) Appl. No.: 18/829,780

(22) Filed: Sep. 10, 2024

(51) Int. Cl. G06F 3/01; H01B 5/16

(52) U.S. Cl. CPC G06F 3/015; A61B 5/293; H01B 3/12; H01B 5/16

(57) ABSTRACT

A method and system for protecting electrode arrays while implanted on a brain including a flexible electrode array including non-penetrating cortical surface microelectrodes, a ceramic layer covering the electrode array and a polymer layer adjacent the ceramic layer, wherein the ceramic layer and the polymer layer hermetically seal the electrode array. The ceramic layer can be configured to exhibit sufficient flexibility to allow the electrode array to be implanted using minimally invasive surgical techniques, while still hermetically sealing the electrode array from a biological environment.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through early phase clinical trials.

High bandwidth brain-computer interfaces are being developed to enable the bidirectional communication between the nervous system and external computer systems in order to assist, augment, or replace neurological function lost to disease or injury. A brain-computer interface should be able to accurately decode electrophysiologic signals recorded from individual neurons, or populations of neurons, and correlate such activity with one or more sensory stimuli or intended motor response. For example, such a system may record activity from the primary motor cortex in an animal or a paralyzed human patient and attempt to predict the actual or intended movement in a specific body part; or the system may record activity from the visual cortex and attempt to predict both the location and nature of the stimuli present in the patient's visual field.

However, neural interfaces generally fail at material interfaces if the neural interface is left implanted on the brain for long periods of time. For example, neural interfaces that have only polymer insulation suffer from corrosion at metal conductors due to the high water vapor transmission rate of polymers. Although the polymer layer does not dissolve when exposed to bodily fluid, it begins to delaminate from the metal electrode array, which opens up the metal to corrosion and causes failures in the wiring and electronics of the neural interface. Conventional neural interfaces do not have the electrode and trace density or the intended implant lifetime for the delamination of the polymer layers to become an issue. Therefore, conventional neural interfaces have not targeted such improvements to barrier layers and insulation. However, there would be highly beneficial use cases if high density, high bandwidth neural interfaces could be left implanted in subjects for extended periods of time. Therefore, layers or barriers capable of increasing interface longevity in biological environments would be highly beneficial.

### SUMMARY

The present disclosure is directed to systems and methods for protecting electrode arrays while implanted on a brain. Specifically, the present disclosure is directed to systems and methods of coating an electrode array to prevent corrosion while implanted.

In some embodiments, there is provided a neural interface, including: a flexible electrode array including non-penetrating cortical surface microelectrodes; at least one ceramic layer covering the electrode array, the at least one ceramic layer having a thickness from about 50 nm to about 1000 nm and a refractive index from about 1.55 to about 1.78; and at least one polymer layer adhered to the at least one ceramic layer; wherein the at least one ceramic layer and the at least one polymer layer insulate the electrode array. In some embodiments, the neural interface further can include a via etched in the ceramic layer.

In some embodiments, the at least one ceramic layer can include silicon oxynitride or silicon carbide.

In some embodiments, the neural interface can further include wherein the polymer layer overlaps the via defined by an edge of the ceramic layer.

In some embodiments, the neural interface can further include wherein the ceramic layer defining the via underlaps an edge of the polymer layer.

In some embodiments, the neural interface can further include wherein the ceramic layer is deposited via plasma enhanced chemical vapor deposition (PECVD). In some embodiments, the neural interface can further include wherein the ceramic layer is deposited via high density plasma enhanced chemical vapor deposition (HDPECVD).

In some embodiments, the neural interface can include a plurality of polymer layers and the polymer layer is one of the plurality of polymer layers.

In some embodiments, the neural interface can include a plurality of ceramic layers and the ceramic layer is one of the plurality of ceramic layers.

In some embodiments, there is provided a method of insulating a neural interface, the method including: providing a polymer electrode array substrate in a deposition chamber; setting a temperature of the deposition chamber to between about 50° C. and about 250° C.; depositing a first ceramic layer onto the polymer electrode array substrate, wherein the first ceramic layer has a deposited thickness of between approximately 50 nm and approximately 1000 nm, and wherein the first ceramic layer has a refractive index from about 1.55 to about 1.78; patterning a metal layer onto the first ceramic layer; depositing a second ceramic layer onto the metal layer, wherein the second ceramic layer is substantially similar to the first ceramic layer; and adhering a polymer layer to at least the second ceramic layer. In some embodiments, the method can further include etching a via in the second ceramic layer. In some embodiments, the method can further include wherein the ceramic layer is deposited via a HDPECVD tool.

In some embodiments, the first ceramic layer and the second ceramic layer can include silicon oxynitride.

In some embodiments, the first ceramic layer and the second ceramic layer can include silicon carbide.

In some embodiments, the method can further include etching a via in the polymer layer.

### FIGURES

FIG. 1 depicts a block diagram of a secure neural device data transfer system, in accordance with an embodiment of the present disclosure.

FIG. 2 depicts a diagram of a neural device, in accordance with an embodiment of the present disclosure.

FIG. 3 depicts a diagram of a thin-film, microelectrode array neural device and implantation method, in accordance with an embodiment of the present disclosure.

FIG. 4A depicts a neural interface including a via defined by an overlapping polymer layer, in accordance with an embodiment of the present disclosure.

FIG. 4B depicts a neural interface including an electrode array covered by a ceramic layer, in accordance with an embodiment of the present disclosure.

FIG. 4C depicts a neural interface including a via defined by an underlapping ceramic layer, in accordance with an embodiment of the present disclosure.

FIG. 4D depicts a system view of a neural interface, in accordance with an embodiment of the present disclosure.

FIG. 5 depicts a diagram of a method of covering an electrode array, in accordance with an embodiment of the present disclosure.

### DETAILED DESCRIPTION

The present disclosure is generally directed to systems and methods for protecting electrode arrays while implanted in the body. In particular, the present disclosure is directed to using silicon oxynitride to cover the electrode on both sides to serve as a barrier against water vapor.

Disclosed herein is a system of protecting an electrode array with a ceramic layer such that the neural device including the electrode array can be placed on the brain for an extended period of time, without the electrode array corroding and causing delamination or dissolution of materials, leading to material exposure to the brain or electrical loss. Embodiments can include the electrode array covered in a layer of silicon oxynitride on both sides of the metal. Embodiments can include the method of covering the electrode array with a ceramic layer.

### Neural Device Systems

Conventional neural devices typically include electrode arrays that penetrate a subject's brain in order to sense and/or stimulate the brain. However, the present disclosure is directed to the use of non-penetrating neural devices, i.e., neural devices having electrode arrays that do not penetrate the cortical surface. Such non-penetrating neural devices are minimally invasive and minimize the amount of impact on the subject's cortical tissue. Neural devices can sense and record brain activity, receive instructions for stimulating the subject's brain, and otherwise interact with a subject's brain as generally described herein.

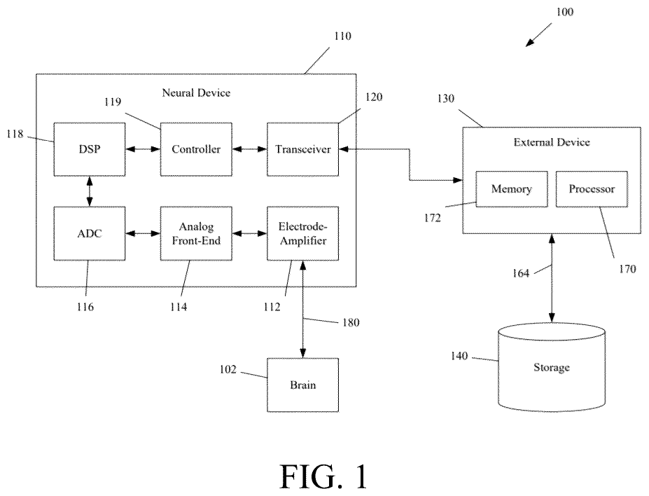

Referring now to FIGS. 1-3, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some embodiments, the electrode array 180 of the neural device 110 can have electrodes that are sufficiently small and spaced at sufficiently small distances in order to define a high-density electrode array 180 that can, accordingly, capture high resolution electrocortical data. Such high-resolution data can be used to resolve electrocorticographic features that can otherwise not be identified using lower resolution electrode arrays. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm.

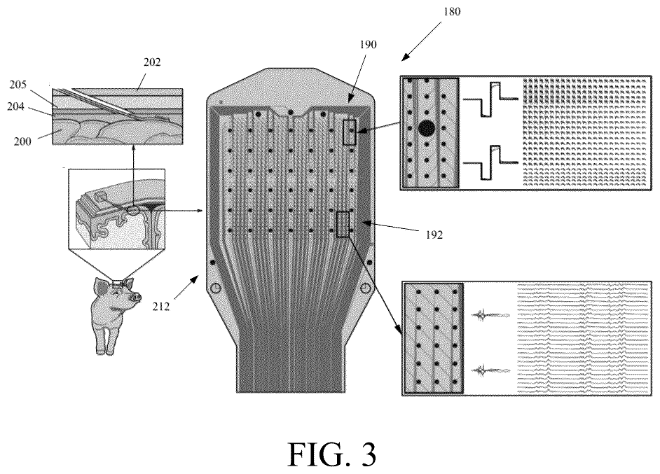

The neural device 110 can further include a flexible substrate 212 supporting the electrode array 180 and/or other components of the neural device 110, as shown in FIG. 3. In some embodiments, the flexible substrate 212 can be flexible enough to permit the electrode array 180 to be inserted through an osteotomy into the subdural space 204, then along the cortical surface.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array 180, such as is described below, that is able to physically interface with the brain 102 of the subject in order to sense brain signals and/or apply electrical signals thereto. The analog front-end stage 114 can be configured, amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject.

In some embodiments, the neural device 110 can include a controller 119 that is configured to perform various functions, including compressing electrophysiologic data generated by the electrode array 180. In various embodiments, the controller 119 can include hardware, software, firmware, or various combinations thereof that are operable to execute the functions described below. In one embodiment, the controller 119 can include a processor (e.g., a microprocessor) executing instructions stored in a memory. In another embodiment, the controller 119 can include a field-programmable gate array (FPGA) or application-specific integrated circuit (ASIC).

In various embodiments, the stages of the neural device 110 can provide unidirectional or bidirectional communications (as indicated in FIG. 1) by and between the neural device 110 and the external device 130. In various embodiments, one or more of the stages can operate in a serial or parallel manner with other stages of the system 100. It can further be noted that the depicted architecture for the system 100 is simply intended for illustrative purposes and that the system 100 can be arranged differently (i.e., components or stages can be connected in different manners) or include additional components or stages.

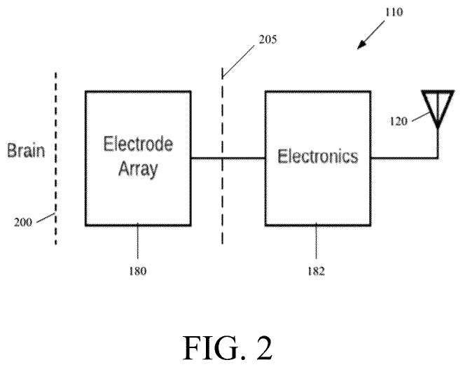

In some embodiments, the neural device 110 described above can include a brain implant, such as is shown in FIG. 2. The neural device 110 can be a biomedical device configured to study, investigate, diagnose, treat, and/or augment brain activity. In some embodiments, the neural device 110 can be positioned between the brain 200 and the scalp 202, as shown in FIG. 3. In some embodiments, the neural device 110 can be positioned between the brain 200 and the dura 205. The neural device 110 can include an electrode array 180 (which can be a component of or coupled to the electrode-amplifier stage 112 described above) that is configured to record and/or stimulate an area of the brain 200. The electrode array 180 can be connected to an electronics hub 182 (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

The electrode array 180 can include non-penetrating cortical surface microelectrodes (i.e., the electrode array 180 does not penetrate the brain 200). Accordingly, the neural device 110 can provide a high spatial resolution, with minimal invasiveness and improved signal quality. The minimal invasiveness of the electrode array 180 is beneficial because it allows the neural device 110 to be used with larger population of patients than conventional brain implants, thereby expanding the application of the neural device 110 and allowing more individuals to benefit from brain-computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features, tighter electrode spacing, and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

Referring now to FIG. 3, there is shown a diagram of an illustrative embodiment of a neural device 110. In this embodiment, the neural device 110 can include an electrode array 180 including nonpenetrating microelectrodes. As generally described above, the neural device 110 is configured for minimally invasive subdural implantation using a cranial micro-slit technique. In some embodiments, the neural device 110 is inserted into the subdural space 204 between the dura 205 and the surface of the brain 200. Further, the microelectrodes of the electrode array 180 can be arranged in a variety of different configurations and can vary in size. In this particular example, the electrode array 180 includes a first group 190 of electrodes (e.g., 200 μm microelectrodes) and a second group 192 of electrodes (e.g., 20 μm microelectrodes). Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 180 provides multichannel data that can be used in a variety of electrophysiologic paradigms to perform neural recording of both spontaneous and stimulus-evoked neural activity as well as decoding and focal stimulation of neural activity across a variety of functional brain regions.

Additional information regarding brain-computer interfaces described herein can be found in U.S. Provisional Patent Application No. 63/621,353, titled MINIMALLY INVASIVE INSERTION SYSTEM FOR NEURAL INTERFACES, filed Jan. 16, 2024; and in Ho et al., The Layer 7 Cortical Interface: A Scalable and Minimally Invasive Brain—Computer Interface Platform, bioRxiv 2022.01.02.474656; doi: https://doi.org/10.1101/2022.01.02.474656, which are hereby incorporated by reference herein in their entireties.

### Hermetic Film for Electrode Array Longevity

Conventional neural interfaces are limited to low electrode density when implanted for long periods of time; however, it would be beneficial if high density neural interfaces could overcome this limitation, such that existing low density interfaces that are designed conservatively could be extended to high density while implanted chronically. Conventional high density neural interfaces fabricated by thin-film methods have polymer layers that encapsulate and seal the electrode array from the biological environment. However, such polymers fail after extended exposure to biological environments and allow moisture through, which in turn causes corrosion of the electrodes in the array. In some instances, the polymer, which can be polyimide, can even delaminate from the electrode array. Corrosion of the electrode array can cause the neural interface to fail. Therefore, polymers used with conventional neural interfaces are not suitable for use with neural interfaces that are intended to be implanted for extended periods of time. One additional issue is that many materials suitable for use in creating hermetic electronics are stiff, which would not be suitable for use with the neural interfaces described herein because such neural interfaces require flexibility because of their interface with soft neural tissue. Therefore, materials are needed that are both capable of being used as hermetic barriers for extended periods of time when exposed to biological environments, while having sufficient flexibility to be usable with the neural interfaces and implantation techniques described herein.

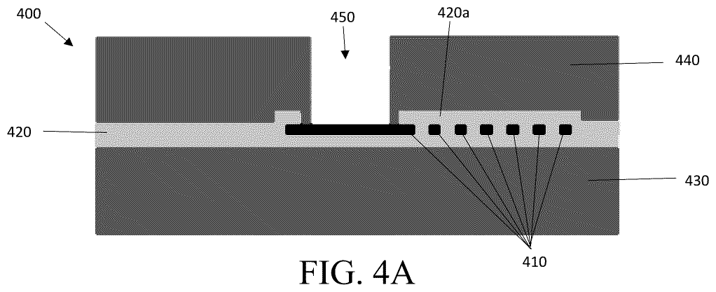

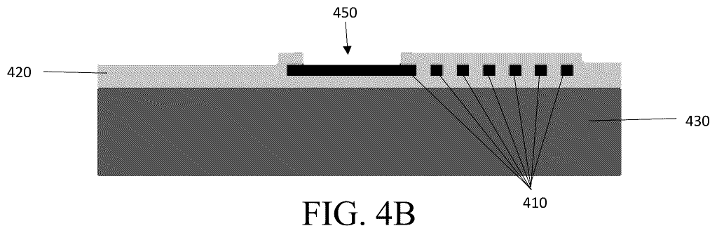

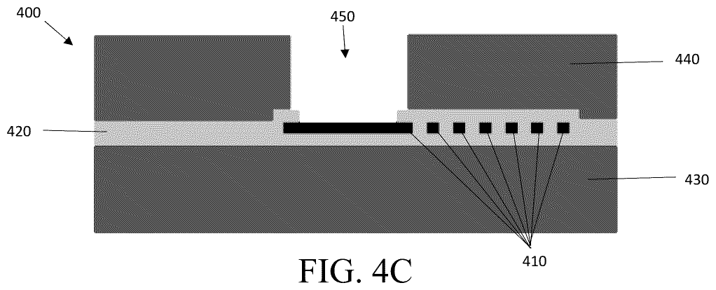

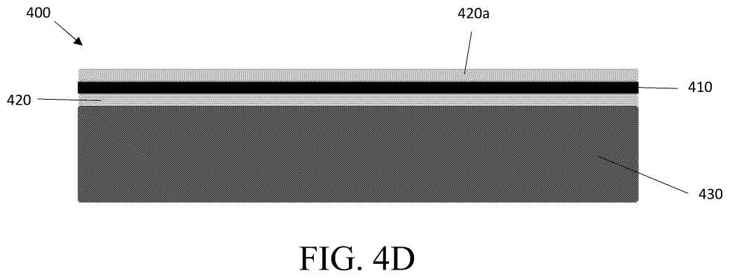

The neural interface can have different configurations of ceramic and polymer. Referring to FIG. 4A, there is shown a neural interface 400 including a via 450 defined by an overlapping polymer layer 440, in accordance with an embodiment of the present disclosure. The neural interface 400 can include an electrode array 410 covered by a ceramic layer 420. The ceramic layer 420 can be deposited on a polymer layer 430. The ceramic layer 420 can be adhered to the polymer layer 430. In some embodiments, the polymer layer 430 can be treated to enhance adhesion of the ceramic layer 420. In some embodiments, there can be one ceramic layer 420 surrounding the electrode array 410. In some embodiments, there can be one ceramic layer 420 on one side of the electrode array 410 and a second ceramic layer 420a on a second side of the electrode array 410. The neural interface can include a second polymer layer 440. In some embodiments, the second polymer layer 440 can be adhered to the ceramic layer 420. In some embodiments, the second polymer layer 440 can be adhered to the second ceramic layer 420a.

The via 450 can be etched in the ceramic layer 420. In some embodiments, the via 450 can be etched in the second ceramic layer 420a. In some embodiments, the via 450 can be etched in the second polymer layer 440. The via 450 can expose a portion of the electrode array 410. The second polymer layer 440 can overlap the ceramic layer 420, such that the via 450 is defined by the second polymer layer 440 and the electrode array 410. In some embodiments, the overlap prevents the ceramic layer 420 from being exposed to bodily fluids.

The electrode array 410 can include a metallic material. In some embodiments, the electrode array 410 can be the electrode array 180. The electrode array 410 can include non-penetrating cortical surface microelectrodes, as with the neural devices 110 described above. In some embodiments, the electrode array 410 can be patterned on the ceramic layer 420 prior to the second ceramic layer 420a being deposited.

The ceramic layer 420 can include a ceramic material. In some embodiments, the ceramic layer 420 can include silicon oxynitride. In some embodiments, the ceramic layer 420 can be deposited on the electrode array. In some embodiments, the ceramic layer 420 can be deposited by plasma enhanced chemical vapor deposition (PECVD). In some embodiments, the ceramic layer 420 can be deposited by high density plasma enhanced chemical vapor deposition (HDPECVD). With both PECVD and HDPECVD the parameters of the film composition can be tuned by adjusting the depositing environment. For example, the gas flow rate, power, pressure, and/or temperature of the depositing environment can be adjusted to target a certain film composition and/or thickness. Using HDPECVD can provide higher stability of silicon oxynitride by deposition than PECVD. Silicon oxynitride's characteristics can allow for a broader composition range while remaining biostable and, in some composition ranges, allow for increased flexibility.

As noted above, it is desirable for the electrode array 180 to maintain flexibility because of the minimally invasive method by which the neural device 110 can be implanted within a subject. During implantation, the neural device can be flexed. In some embodiments, the neural device can be implanted by a minimally invasive insertion method. In some embodiments, the neural device can be implanted through a narrow osteotomy slit. In some embodiments, in order to maintain flexibility, the ceramic layer 420 can be deposited in one or more thin layers. Embodiments where the ceramic layer 420 includes silicon oxynitride can be beneficial because, it is flexible when deposited within a particular thickness range, while still maintaining its hermetic properties. If the ceramic layer 420 is too thick, then the ceramic layer 420 will start off with higher stress and stiffness, which can lead to quicker failure of the neural device. With high stiffness, the ceramic layer 420 may be more resistant to flexing than with a lower stiffness. Therefore, it is desirable to balance the thickness of the silicon oxynitride layer so that it is not too thin to serve as an effective hermetic barrier, but also not too thick such that it is insufficiently flexible. In some embodiments, the ceramic layer 420 can be deposited to a thickness of about 50 nm to about 1000 nm. In some embodiments, the ceramic layer 420 can be deposited to a thickness of about 50 nm to about 500 nm.

As noted above, in some embodiments, the ceramic layer 420 can include silicon oxynitride. The ability of silicon oxynitride to serve as a hermetic barrier is based on its composition, i.e., the ratio of nitrogen to oxygen within the silicon oxynitride. As a person having ordinary skill in the art will understand, a proxy for determining the composition is to measure the refractive index of the silicon oxynitride, which can be determined by using an ellipsometer or other optical metrology tool. Accordingly, the refractive index of the silicon oxynitride can be used as a correlate for the hermetic properties of a silicon oxynitride film layer. Therefore, in some embodiments, the refractive index of the silicon oxynitride layer can be controlled during manufacture to control the hermetic properties of the ceramic layer 420. In some embodiments, the refractive index of the silicon oxynitride layer can be about 1.45, 1.5, 1.55, 1.6, 1.65, 1.7, 1.75, 1.8, 1.85, 1.9, 1.95, 2.0, or any range there between, including endpoints. In one illustrative embodiment, the silicon oxynitride layer can have a refractive index of about 1.55 to about 1.78. In this range of material composition, instead of dissolving, as some other ceramics do, silicon oxynitride is biostable. Due to the nature of ceramics, the stiffness of silicon oxynitride increases as the thickness increases. Further, the material composition described herein for silicon oxynitride exhibits a low water vapor transmission rate, high biostability, and low stress, thereby allowing the ceramic layer 420 to serve as a hermetic barrier with a biological environment for an extended period of time. Therefore, in thin layers and with a refractive index within the ranges described herein, the silicon oxynitride can be both a barrier and flexible enough while implanted on the brain.

Unlike silicon oxynitride, not all ceramic films exhibit high biostability, including silicon oxide and silicon nitride, which dissolve over time. Silicon carbide, another common highly stable barrier material, relies on tight stoichiometric control for its stability and is strongly dependent on deposition parameters for surface structure and adhesion. Silicon carbide is also difficult to deposit and etch in production environments with reliable protective properties and electrode surface exposure. In contrast, silicon oxynitride has a range of acceptable compositions for biostability and film quality and can be tightly tracked by determining the refractive index. Further, the chemical properties of silicon oxynitride allow for more straightforward bonding to certain metals and metallic compounds than some other ceramics.

The polymer layer 430 can be adhered to the ceramic layer 420. In some embodiments, there can be a second polymer layer 440 adhered to the ceramic layer 420. The second polymer layer 440 can be adhered to a side of the ceramic layer 420 opposite the polymer layer 430. In some embodiments, the second polymer layer 440 can be adhered to the second ceramic layer 420a.

Referring now to FIG. 4B, there is shown a neural interface 400 including an electrode array 410 covered by a ceramic layer 420, in accordance with an embodiment of the present disclosure. In some embodiments, the neural interface 400 can include the polymer layer 430, the ceramic layer 420, and the electrode array 410. The ceramic layer 420 can cover the electrode array 410. In some embodiments, a second polymer layer 440 can be adhered to the ceramic layer 420. In some embodiments, the polymer layers 430, 440 can include polyimides, parylene, and/or liquid crystal polymers. In some embodiments, the polymer layer can be treated by plasma and/or chemical means to modify the surface and/or to increase adhesion between the polymer layers 430, 440 and the ceramic layer 420. In some embodiments, the ceramic layer 420 can be treated by plasma and/or chemical means to increase adhesion between the ceramic layer 420 and the polymer layers 430, 440. In some embodiments, the treatment can include aminosilane and/or other silane treatments. The polymer layer 430 can insulate the ceramic layer 420 which in turn insulates the electrode array 410. Thus, the electrode array 410 can have two levels of insulation from bodily fluids to decrease the likelihood of bodily fluids corroding it and increase the longevity of the electrode array 410.

Referring now to FIG. 4C, there is shown a neural interface 400 including a via 450 defined by an underlapping ceramic layer 420, in accordance with an embodiment of the present disclosure. The ceramic layer 420 can be deposited on a polymer layer 430. In some embodiments, the ceramic layer 420 can be adhered to the polymer layer 430. The polymer layer 430 can be treated to enhance adhesion of the ceramic layer 420. In some embodiments, a second polymer layer 440 can be adhered to the ceramic layer 420. A via 450 can be etched in the ceramic layer 420. In some embodiments, the via 450 can be etched in the second polymer layer 440. In some embodiments, the via 450 can be defined by the ceramic layer 420 and the electrode array 410. The second polymer layer 440 can be away from the edge of the via 450 such that an underlap is created allowing the ceramic layer 420 to be the only layer in contact with the electrode array 410. The second polymer layer 440 not being in contact with the electrode array 410 can allow for less delamination, as the bond between the ceramic layer 420 and the electrode array 410 can in some embodiments be stronger than the bond between the second polymer layer 440 and the electrode array 410.

Referring now to FIG. 4D, there is shown a system view of the neural interface 400, in accordance with an embodiment of the present disclosure. In some embodiments, the neural interface 400 can include a polymer layer 430, a ceramic layer 420, an electrode array 410, and a second ceramic layer 420a. The ceramic layer 420 and the second ceramic layer 420a can be on opposing sides of the electrode array 410 such that the electrode array 410 is sandwiched and insulated on both sides. In some embodiments, a second polymer layer (not shown) can be adhered to the second ceramic layer 420a, similarly to the embodiment shown in FIG. 4C. This can be a general system view of the layers, and the details of the layers can be adjusted. As one skilled in the art will understand, the layers can not be to scale, and the thickness of each layer can vary.

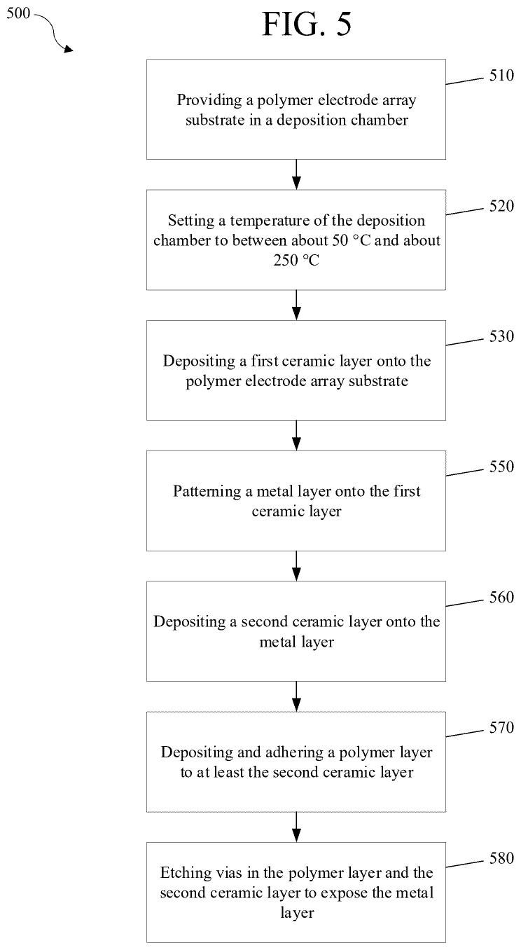

Referring now to FIG. 5, there is shown a method of insulating a neural interface. In step 510, an electrode array substrate is provided in a deposition chamber. In some embodiments, the electrode array substrate can include polymer. In some implementations, the electrode array substrate can be treated chemically or by plasma prior to step 510, as described throughout the present disclosure. In some embodiments, the electrode array substrate can be the polymer layer 430. In step 520, a temperature is set in the deposition chamber to between about 20° C. and about 250° C. In some embodiments, the deposition chamber can be part of a HDPECVD tool. In some embodiments, the electrode array substrate can be set to a temperature different than the temperature of the deposition chamber. In some embodiments, the temperature of the electrode array substrate can be set to a temperature higher than the temperature in the deposition chamber.

In step 530, a first ceramic layer can be deposited onto the electrode array substrate. In some embodiments, the first ceramic layer can include silicon oxynitride. In some embodiments, the first ceramic layer can be the ceramic layer 420. The ceramic layer can have a refractive index between about 1.55 and about 1.78 and can be deposited to a thickness of about 50 nm to about 1000 nm. In some embodiments, the ceramic layer can be deposited by HDPECVD.

HDPECVD can utilize an inductively coupled plasma (ICP) power generator which leads to higher density plasma that operates at a lower pressure than PECVD and increases precursor gas decomposition that leads to higher film quality than PECVD is able to provide. In addition to the ICP power generator, HDPECVD can utilize a radio frequency (RF) plasma generator. The PECVD process can be achieved with RF power between about 50 W to about 250 W, a pressure between about 600 mTorr and about 1400 mTorr, and a temperature between about 250° C. to about 350° C. In contrast, the HDPECVD process can be achieved with an RF bias power between about OW to about 150 W, an ICP power between about 600 W to about 2500 W, and a pressure of about 5 mTorr to about 50 mTorr. The HDPECVD process provides high density plasma by which the chemicals injected to be deposited in the deposition chamber are decomposed efficiently into reactants. When chemical gases are injected into the deposition chamber, the high density plasma decomposes the gas and provides energy for chemical reactions to occur on surfaces using the products of the decomposition. In some embodiments, the gas can be a reactive gas. In some embodiments, the gas can include at least one of silane gas, nitrous oxide gas, ammonia gas, methane gas, nitrogen gas, oxygen gas, argon gas, and/or hydrogen gas. In some embodiments, the gas can include a combination of silane gas, nitrogen gas, and oxygen gas. In some embodiments, the gas can include a combination of silane gas, nitrous oxide gas, and ammonia gas. In some embodiments, the flow rates of the different gases can be varied such that the gases are injected into the chamber at different rates.

In some embodiments, the HDPECVD tool can include RF plasma generators and/or low frequency (LF) plasma generators. The RF and LF plasma generators can be pulsed to achieve modified, and in some cases lower, stress and/or stiffness of the silicon oxynitride film. In addition, chamber pressure and ICP power can affect both the stress and refractive index of the silicon oxynitride film. Various factors can be optimized to target a refractive index while optimizing the stress of the film.

The HDPECVD process can operate at lower temperatures than other chemical vapor deposition processes. HDPECVD can deposit high-quality films at room temperature and lower temperatures than PECVD in general. For example, PECVD requires temperatures between about 250° C. and about 350° C. In contrast, HDPECVD can operate at temperatures between about 20° C. and about 250° C. In some embodiments, the silicon oxynitride layer can be deposited between about 50° C. and about 250° C. In some embodiments, the silicon oxynitride layer can be deposited at a temperature between about 50° C. and about 150° C. While using lower temperatures than PECVD, HDPECVD can achieve high uniformity of the film and beneficial film properties which usually require minimum threshold temperatures.

In some embodiments, the ceramic layer can be treated by plasma and/or chemical treatment. In some embodiments, the plasma and/or chemical treatment can include aminosilane and/or silane treatments. In some embodiments, the first ceramic layer can be treated prior to patterning the electrode array to provide adhesion to the electrode array. In some embodiments, the second ceramic layer can be treated prior to adding the polymer layer to provide stronger adhesion to the polymer layer. In other embodiments, the process 500 can omit the step of treating the ceramic layer with a plasma and/or chemical treatment.

In step 550, metal can be patterned onto the ceramic layer to form the electrode array. In some embodiments, the metal layer can include non-penetrating cortical microelectrodes. In some embodiments, the metal layer can include the electrode array 410. In some embodiments, the electrode array 410 can be inserted into the deposition chamber after deposition of the first ceramic layer.

In step 560, a second ceramic layer can be deposited onto the metal layer. The second ceramic layer can be substantially similar to the first ceramic layer. In some embodiments, the second ceramic layer can be deposited by the same method as described in step 530. In some embodiments, the second ceramic layer can include silicon oxynitride. In some embodiments, the second ceramic layer can include silicon carbide. The second ceramic layer can be the second ceramic layer 420a. In some embodiments, the second ceramic layer can be treated by plasma and/or chemical treatment in substantially the same method as described in step 540.

In step 570, a polymer layer can be adhered to at least the second ceramic layer. In some embodiments, a first polymer layer can be adhered to the electrode array substrate prior to the ceramic layer being deposited in step 530. In some embodiments, the first polymer layer can be adhered to the first ceramic layer after the ceramic layers have been deposited. The first polymer layer can be the polymer layer 430. In some embodiments, a second polymer layer can be adhered to the second ceramic layer. The second polymer layer can be the second polymer layer 440. In some embodiments, the form of the neural device can be in a sandwich pattern including a first polymer layer, a first ceramic layer, an electrode array, a second ceramic layer, and a second polymer layer.

A via can be etched into at least the second ceramic layer 420a, as shown in FIG. 4C. In some embodiments, the via can be etched into the second polymer layer 440, as shown in FIG. 4D. In step 580 of the depicted embodiment of the process 500, vias can be etched in the polymer layer and the second ceramic layer to expose the metal later. In some embodiments, the second polymer layer can overlap the via defined by the second ceramic layer, such that the via can be defined by the second polymer layer and the electrode array. In some embodiments, the second ceramic layer can underlap the second polymer layer, such that the via is defined by the second ceramic layer and the electrode array.

This disclosure is not limited to the particular systems, devices, and methods described, as these can vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “underlap” means that an edge of the silicon oxynitride layer extends beyond an edge of the polymer layer.

As used herein, the term “overlap” means that an edge of the polymer layer extends beyond an edge of the silicon oxynitride layer.

As used herein, the term “stiffness” means the internal resistance of a material to the distorting effects of an external force or load.

As used herein, the term “stress” means the intrinsic quality of a film that exerts a force on other films around it.

As used herein, the singular forms “a,” “an,” and “the” include plural references, unless the context clearly dictates otherwise. Thus, for example, reference to a “layer” is a reference to one or more layers and equivalents thereof known to those skilled in the art, and so forth.

As used herein, the term “about” means plus or minus 10% of the numerical value of the number with which it is being used. Therefore, about 50 nm means in the range of 45 nm to 55 nm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures can be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, can be combined into many other different systems or applications. Various presently unforeseen or unanticipated alternatives, modifications, variations or improvements therein may be subsequently made by those skilled in the art, each of which is also intended to be encompassed by the disclosed embodiments.

1. A neural interface, comprising:
a flexible electrode array comprising non-penetrating cortical surface microelectrodes;
at least one ceramic layer covering the electrode array, the at least one ceramic layer comprising a thickness from about 50 nm to about 1000 nm and a refractive index from about 1.55 to about 1.78; and
at least one polymer layer adhered to the at least one ceramic layer, the at least one polymer layer covering at least a portion of the at least one ceramic layer;
wherein the at least one ceramic layer and the at least one polymer layer seal the electrode array,
wherein the at least one polymer layer comprises a polymer comprising at least one of polyimide, parylene, and liquid crystal polymer, and
wherein the at least one ceramic layer is comprised of silicon oxynitride or silicon carbide.

2. (canceled)

3. The neural interface of claim 1, further comprising:
a via etched in the ceramic layer.

4. The neural interface of claim 3, wherein the polymer layer's via edge overlaps the via defined by an edge of the ceramic layer.

5. The neural interface of claim 3, wherein the ceramic layer defining the via underlaps an edge of the polymer layer.

6. The neural interface of claim 1, wherein the ceramic layer is a first ceramic layer on a first side of the electrode array and wherein the polymer layer is a first polymer layer on the first side of the electrode array, the neural interface further comprising:
a second ceramic layer covering the electrode array on a second side of the electrode array opposite the first side of the electrode array, such that the first ceramic layer and the second ceramic layer sandwich the electrode array; and
a second polymer layer adhered to the second ceramic layer on the second side of the electrode array.

7. The neural interface of claim 1, wherein the ceramic layer is deposited via high density plasma enhanced chemical vapor deposition (HDPECVD).

8. The neural interface of claim 7, wherein the HDPECVD comprises a pulsed radio frequency plasma generator and an inductively coupled plasma generator.

9. A method of insulating a neural interface, the method comprising:
providing a polymer electrode array substrate in a deposition chamber;
setting a temperature of the deposition chamber to between about 50° C. and about 250° C.;
depositing a first ceramic layer onto the polymer electrode array substrate, wherein the first ceramic layer has a deposited thickness of between about 50 nm and about 1000 nm, and wherein the first ceramic layer has a refractive index from about 1.55 to about 1.78;
patterning a metal layer onto the first ceramic layer, the metal layer comprising non-penetrating cortical surface microelectrodes;
depositing a second ceramic layer onto the metal layer, wherein the second ceramic layer is substantially similar to the first ceramic layer; and
adhering a polymer layer to at least the second ceramic layer, the at least one polymer layer covering at least a portion of the at least one ceramic layer,
wherein the at least one polymer layer comprises a polymer comprising at least one of polyimide, parylene, and liquid crystal polymer, and
wherein the first ceramic layer and the second ceramic layer comprise at least one of silicon oxynitride or silicon carbide.

10. The method of claim 9, further comprising:
etching a via in the second polymer and ceramic layers.

11. The method of claim 10, wherein the polymer layer is adhered to the second ceramic layer such that the polymer layer overlaps the via defined by an edge of the second ceramic layer.

12. The method of claim 10, wherein the polymer layer is adhered to the second ceramic layer such that the second ceramic layer defining the via underlaps an edge of the polymer layer.

13-14. (canceled)

15. The method of claim 9, wherein the set temperature is between about 50° C. and about 120° C.

16. The method of claim 9, wherein the first ceramic layer and the second ceramic layer are deposited via a HDPECVD tool.

17. The method of claim 16, wherein the HDPECVD tool operates at a pressure between about 5 mTorr and about 50 mTorr.

18. The method of claim 16, wherein the HDPECVD tool comprises a radio frequency plasma generator and an inductively coupled plasma generator, wherein the radio frequency plasma generator is pulsed during operation of the HDPECVD tool.

19. The method of claim 18, wherein the radio frequency plasma generator is pulsed between about 0 W and about 150 W.

20. The method of claim 9, wherein the plasma or chemical treatment of the ceramic layer comprises at least one of aminosilane or silane treatments.
